(19) United States
(12) Patent Application Publication
(10) Pub. No.: US 2007/0213253 A1
(43) Pub. Date: Sep. 13, 2007

(54) Synuclein Mutant Having Aggregation-Inhibitory Activity

(76) Inventor: Koji Sode, Meguro-ku (JP)

Correspondence Address: BIRCH STEWART KOLASCH & BIRCH, PO BOX 747, FALLS CHURCH, VA, 22040-0747, US

(21) Appl. No.: 10/562,063

(22) PCT Filed: Jun. 22, 2004
(86) PCT No.: PCT/JP04/09084

§ 371 Date: Jan. 19, 2007

(30) Foreign Application Priority Data
Jun. 22, 2003 (JP) 2003-202699

(51) Int. Cl. C07K 14/47; C07K 7/06; C12N 1/15; C12N 1/19; C12N 1/21; C12N 15/12; C12N 5/10; C12P 21/02

(52) U.S. Cl. 514/2; 435/320.1; 435/325; 435/69.1; 530/350; 536/23.5

(57) ABSTRACT

Disclosed is a mutant human α-synuclein with decreased ability of forming aggregation. The mutant human α-synuclein of the invention is able to inhibit aggregation of the wild type human α-synuclein, Ala53Thr mutant human α-synuclein or Ala50Pro mutant human α-synuclein, thus is useful for investigation of pathology and treatment of Parkinson's disease and for research and development of gene therapy. Also disclosed is a partial structure peptide of human α-synuclein comprising amino acid substitutions as taught by the invention.

### TECHNICAL FIELD

The present invention relates to a novel mutant human α-synuclein protein.

### BACKGROUND ART

α-Synuclein is a heat-stable protein consisting of 140 amino acid residues. Since accumulation of coagulate of α-synuclein is noted in Lewy body in the brain of patients suffering from Parkinson's disease, researches have been focused on the relation between accumulation of abnormal proteins and nerve cell death, as experienced in various neurodegenerative diseases. It has been said that α-synuclein does not form a specific steric structure in vivo and belongs to a natively unfolded protein family.

α-Synuclein is divided into three regions based on its primary structure. The central region comprising 35 amino acid residues is NAC (Non-amyloid β-component of Alzheimer's disease amyloid) which is the second constituting component of senile plaque noted in the brain of patients suffering from Alzheimer's disease. It has been shown that this region has a high ability for formation of β-sheet and is particularly involved in aggregation (Ueda K, Fukushima H, Masliah E, Xia Y, Iwai A, Yoshimoto M, Otero D A, Kondo J, Ihara, Y, Saitoh T. Proc. Natl. Acad. Sci. U.S.A. 1993; 90(23):11282-6., Iwai A, Yoshimoto M, Masliah E, Saitoh T., Biochemistry. 1995; 34(32):10139-45., Han H, Weinreb P H, Lansbury P T Jr. Chem. Biol. 1995 (3):163-9.).

It has also been suggested that aggregation of synuclein is promoted by point mutations Ala50Pro and Ala53Thr at the positions upstream from the NAC region, which are hereditary point mutations noted in familial Parkinson's disease (Linda Narhi, Stephen J. Wood, Shirley Steavenson, Yijia Jiang, Gay May Wu, Dan Anafi, Stephen A, Kaufman, Francis Martin, Karen Sitney, Paul Denis, Jean-Claude Louis, Jette Wypych, Anja Leona Biere and Martin Citron, J. Biol. Chem., 1999; 274: 9843-9846., Rochet J-C, Conway K. A, Lansbury P. T, Biochemistry (2000) 39, 10619-626., Conway K. A, Harper J. D, Lansbury P. T, Nature Medicine (1998) 4, 1318-1320., Li. J., Uversky V. N, Fink A. L, Biochemistry (2001) 40, 11604-613). However, up to now, the mechanisms for aggregation and fibril formation of α-synuclein have not been elucidated through protein-chemical analysis on the basis of systematic construction of mutant α-synucleins.

An object of the present invention is to provide a mutant human α-synuclein capable of inhibiting aggregation of the wild type human α-synuclein.

### DISCLOSURE OF THE INVENTION

The present inventor has conducted various investigations on amino acid residues responsible for aggregation of α-synuclein and found a mutant human α-synuclein with a lower ability of forming aggregation.

The present invention provides a mutant human α-synuclein with decreased ability of forming aggregation.

More particularly, the present invention provides a mutant human α-synuclein having the amino acid sequence where at least one of the following amino acid residues is substituted in the amino acid sequence (SEQ ID NO: 1) of the wild-type human α-synuclein: Gly68; Ala69; Val70; Val71; Thr72; Val74; Val77; and Val82.

Preferably, the mutant human α-synuclein of the present invention contains at least one of the following amino acid substitutions in the amino acid sequence set forth in SEQ ID NO: 1: Gly68 with threonine or valine; Ala69 with threonine, valine or lysine; Val70 with threonine, proline or phenylalanine; Val71 with threonine or lysine; Thr72 with valine or glutamic acid; Val74 with threonine; Val77 with threonine; and Val82 with lysine. It is also preferred that the mutant human α-synuclein of the present invention has amino acid substitutions at four positions of Ala69Lys/Val70Thr/Val71Lys/Thr72Glu in the amino acid sequence set forth in SEQ ID NO: 1. It is also preferred that the mutant human α-synuclein of the present invention has amino acid substitutions at five positions of Ala69Lys/Val70Thr/Val71Lys/Thr72Glu and Val82Lys in the amino acid sequence set forth in SEQ ID NO: 1.

In another aspect, the present invention provides a gene coding for the mutant human α-synuclein of the invention, a recombinant plasmid comprising the gene introduced therein, and a transformant transformed with the recombinant plasmid.

The present invention also provides a process for producing a mutant human α-synuclein comprising the steps of:

(a) introducing the gene coding for the mutant human α-synuclein into a plasmid to prepare a recombinant plasmid;

(b) transforming a host with the recombinant plasmid of (a) to prepare a transformant; and

(c) culturing the transformant of (b) to produce the mutant human α-synuclein.

In still another aspect, the present invention provides a peptide having a sequence of 10 or more contiguous amino acid residues of the following amino acid sequence:

(SEQ ID NO: 22)

Gln-Val-Thr-Asn-Val-Gly-Gly-Ala-Thr-Thr-Thr-Gly-
Val-Thr-Ala-Val-Ala-Gln.

Preferably, the peptide of the present invention has the following amino acid sequence:

(SEQ ID NO: 23)

Val-Gly-Gly-Ala-Thr-Thr-Thr-Gly-Val-Thr

In still another aspect, the present invention provides a composition for inhibiting aggregation of the wild type human α-synuclein, Ala53Thr mutant human α-synuclein or Ala50Pro mutant human α-synuclein comprising the mutant human α-synuclein of the present invention or the peptide of the present invention. The present invention further provides a method for inhibiting aggregation of the wild type human α-synuclein, Ala53Thr mutant human α-synuclein or Ala50Pro mutant human α-synuclein in a cell, tissue or organism, comprising contacting the cell, tissue or organism with the mutant human α-synuclein of the present invention or the peptide of the present invention.

### BRIEF DESCRIPTION OF THE DRAWINGS

FIG. 1 shows a time course of fibril formation of the wild type and A53T mutant α-synucleins (WT: wild type α-synuclein; A53T: Ala53Thr mutant α-synuclein; A30P: Ala30Pro mutant α-synuclein).

FIG. 2 shows a time course of fibril formation of the wild type α-synuclein and the mutant α-synuclein of the present invention (WT: wild type α-synuclein; V70T: Val70Thr mutant α-synuclein; V70P: Val70Pro mutant α-synuclein; V70T/V71T: Val70Thr/Val71Thr double mutant α-synuclein).

FIG. 3 is a graph showing formation of aggregate from the wild type α-synuclein and the mutant α-synuclein, and a mixed sample of the wile type or A53T mutant α-synuclein with the mutant α-synuclein of the present invention (before: initial value; after: value after 145 hours; WT: wild type; V70P: Val70Pro mutant; V70T/V71T: Val70Thr/Val71Thr double mutant; A53T: Ala53Thr mutant; WT×V70T/V71T: a mixed sample of wild type with Val70Thr/Val71Thr double mutant; A53T×V70T/V71T: a mixed sample of Ala53Thr with Val70Thr/Val71Thr double mutant).

FIG. 4 is a graph showing a time course of fibril formation of the wild type α-synuclein and the mutant α-synuclein, and of a mixed sample of the wild type or A53T mutant α-synuclein with the mutant α-synuclein of the present invention (WT×V70T/V71T: a mixed sample of wild type α-synuclein with Val70Thr/Val71Thr double mutant; A53T×V70T/V71T: a mixed sample of Ala53Thr mutant α-synuclein with Val70Thr/Val71Thr double mutant).

### DETAILED DESCRIPTION OF THE INVENTION

The mutant human α-synuclein of the present invention may be prepared from a gene coding for the wild type human α-synuclein by genetic engineering techniques. The amino acid sequence of the wild type human α-synuclein and the nucleotide sequence of the gene coding for the protein are shown in SEQ ID NO: 1 and NO: 2, respectively.

First, the nucleotide sequence of the site to be mutated of the gene coding for the wild type human α-synuclein is altered to a nucleotide sequence corresponding to the target amino acid residue using a site-specific mutagenesis to prepare a mutant human α-synuclein gene. The site-specific mutagenesis involves synthesizing a gene coding for the mutant protein using a single-stranded DNA from a plasmid containing the wild type gene as a template and using a synthetic oligonucleotide containing the nucleotide sequence to be mutated as a primer. The reaction may be carried out using any of commercially available kits (Takara Mutan Express Km, etc.).

In the process of the present invention, an oligonucleotide is chemically synthesized, which will be annealed to a single strand of the wild type human α-synuclein gene but is different in the nucleotide sequence corresponding to the site to be substituted. Then the mutant human α-synuclein gene may be synthesized using the synthesized oligonucleotide as a primer and a single-stranded DNA from a plasmid containing the wild type human α-synuclein gene as a template.

Next, the gene coding for the mutant human α-synuclein is inserted into an expression vector to obtain a host vector system for expression. Examples of the host organism used in the present invention may include, but not limited to, Escherichia coli, yeast and Bacillus subtilis.

The peptide of the present invention may also be prepared by solid-phase or liquid-phase peptide synthesizing techniques commonly used in the art.

The aggregation forming ability of the mutant human α-synuclein of the present invention may be measured by a method commonly used for monitoring fibril formation caused by aggregation of a protein, such as amyloid. For example, α-synuclein is prepared in a solution in about 2 mg/ml and incubated at 37° C., and aliquot is collected in a certain intervals. To the collected sample is added a buffer solution of 10 mM Tris-HCl, pH 7.4, containing a fluorescent dye thioflavine T (TfT), which specifically binds to a fibril structure, in a final concentration of 25 μM to make a volume of 100 μl, and fluorescence spectrum is monitored immediately (Ex 440 nm; Em 450-550 nm). The fibril forming rate may be measured by following the increase in fluorescence intensity of TfT.

The inhibitory activity of the mutant human α-synuclein of the invention and the peptide of the invention on aggregation formation of the wild type α-synuclein or two types of mutant α-synucleins—Ala30Pro and Ala53Thr—found in patients suffering from familial Parkinson's disease may be determined by measuring the fibril forming rate of a sample comprising both these human α-synucleins and the mutant human α-synuclein of the present invention.

The mutant human α-synuclein and the peptide identified in the present invention are expected to be useful for repressing progress of synucleopathy, which is a neurodegenerative disease, such as Parkinson's disease, characterized by deposition of Lewy body. More specifically, the therapeutic effect will be achieved by directly administering the mutant human α-synuclein or the peptide of the present invention to the diseased lesion, by permanently or transiently expressing an expression vector containing a structural gene coding for the mutant or the peptide at the diseased lesion, or by coupling the mutant human α-synuclein or the peptide either genetically or chemically with a peptide residue called as protein transduction domain (PTD), which confers cell permeation property, and administering it to the area near the diseased lesion to allow for absorption.

All patents and reference documents explicitly cited in the present specification are entirely incorporated herein by reference. Also all the contents disclosed in the specification and the drawings of the Japanese Patent Application No. 2003-202699, on which the application claims priority, are entirely incorporated herein by reference.

### EXAMPLES

The present invention will be illustrated in more detail with reference to the Examples below. However, the scope of the present invention is not limited to these Examples.

### Example 1

Preparation of Mutant Human α-Synuclein Gene

Cloning of Human α-Synuclein Gene

pTYB1 was utilized as a vector for expression in Escherichia coli. A primer containing NdeI site and a primer containing KpnI site and a partial sequence of the structural gene of intein were used in PCR on a human bone marrow cDNA library to amplify the structural gene of α-synuclein derived from human.

PCR forward primer:

(SEQ ID NO: 3)

5′-CGC CAT ATG GAT GTA TTC ATG AAA GGA CTT TCA
AAG G-3′

PCR reverse primer:
(SEQ ID NO: 4)

5′-GGT ACC CTT GGC AAA GCA GGC TTC AGG TTC GTA
GTC TTG ATA-3′

The reaction condition for PCR was 35 cycles of denaturation at 95° C. (for 1 minute), annealing at 55° C. for 1 minute and elongation at 72° C. for 1 minute. The PCR product was electrophoresed on agarose gel. DNA was purified using Gene Clean II Kit (Bio 101) and subcloned into pGEM-T. Escherichia coli DH5α-MCR was transformed with the plasmid and subjected to a color selection on a plate containing LB/ampicillin (100 g/ml)/IPTG (0.5 mM)/X-Gal (80 μg/ml). The resulting white colonies were grown and the plasmid was extracted and analyzed for the DNA sequence. Colonies having a plasmid with the insertion of α-synuclein structural gene were grown, and the plasmid was extracted and digested with restriction enzymes NdeI and KpnI. The resulting DNA fragments were purified by the same manner as above, and cloned into the expression vector pTYB1, which has been treated with the same restriction enzymes, to construct a vector pTYB1/α-syn. This vector will express a fused protein in which the intein-chitin binding domain is linked to the C terminal of α-synuclein.

After cultivation of Escherichia coli DH5α-MCR transformed with this plasmid, the plasmid was extracted and analyzed for the DNA sequence to confirm that no mutation was introduced.

Site-Specific Mutagenesis

The plasmid containing α-synuclein gene inserted into a cloning vector pGEM-T was used as a template in PCR using primers containing NcoI and PstI restriction sites to amplify the α-synuclein gene fragment.

PCR forward primer-1: Primer containing NcoI site

(SEQ ID NO: 5)

5′-CCA TGG ATG TAT TCA TGA AAG GAC TTT CAA AGG
CCA-3′
PCR reverse primer-2: Primer containing PstI site

(SEQ ID NO: 6)

5′-CCT GCA GTA TTT CTT AGG CTT CAG GTT CGT AGT
CTT G-3′

The amplified fragments were TA cloned and cut out by digestion with restriction enzymes NcoI and PstI. It was ligated to the similarly-treated expression vector pTrc99A to prepare pTrc99A/αsyn. The plasmid was used in PCR using primers containing HindIII-NdeI and KpnI sites to amplify the α-synuclein gene fragment.

PCR forward primer-3: Primer containing HindIIT-
NdeI sites

(SEQ ID NO: 7)

5′-CCAAGCTTCATATGGATGTATTCATGAAAGGACTTT-3′
PCR reverse primer-4: Primer containing KpnI site

(SEQ ID NO: 8)

5′-GGT ACC CTT GGC AAA GCA GGC TTC AGG TTC GTA GTC
TTG ATA-3′

The amplified fragment was TA cloned and cut out by digestion with restriction enzymes HindIII and KpnI. It was ligated to the similarly-treated vector pKF19k for introducing a mutation to prepare pKF19k/αsyn. Escherichia coli HD5α was transformed with this plasmid, then the plasmid was extracted and the nucleotide sequence was confirmed. Mutation was introduced into the α-synuclein gene using Takara Mutan Super Express Km kit using an oligonucleotide for introduction of mutation (shown below) to prepare plasmids each containing the mutant gene (hereinafter generally called as pKF19k/mutant αsyn). In the case that two or more mutations were introduced, these oligonucleotides were appropriately used in combination. Escherichia coli MV1184 strain was transformed with the plasmid, and introduction of mutation was confirmed by sequence analysis.

Oligonucleotides for introduction of mutation:

(SEQ ID NO: 9)

G68T 5′-CAAATGTTGGAACAGCAGTGGTGAC-3′

(SEQ ID NO: 10)

G6SV 5′-CAAATGTTGGAGTGGCAGTGGTGAC-3′

(SEQ ID NO: 11)

A69T 5′-GTTGGAGGAACAGTGGTGACGGG-3′

(SEQ ID NO: 12)

A69V 5′-GTTGGAGGAGTGGTGGTGACGGG-3′

(SEQ ID NO: 13)

C7OT 5′-GGAGGAGCAACAGTGACGGGTG-3′

(SEQ ID NO: 14)

V7OP 5′-GGAGGAGCACCTGTGACGGGTG-3′

(SEQ ID NO: 15)

V7OF 5′-GGAGGAGCATTTGTGACGGGTG-3′

(SEQ ID NO: 16)

V7OT/V71T 5′-CAAATGTTGGAGGAGCAACAACAACGGGTGTGACAGC
AG-3′

(SEQ ID NO: 17)

T72V 5′-GAGCAGTGGTGGTGGGTGTGACAG-3′

(SEQ ID NO: 18)

V74T 5′-GGTGACGGGTACAACAGCAGTAG-3′

(SEQ ID NO: 19)

V77T 5′-GTGTGACAGCAACCGCCCAGAAGAC-3′

(SEQ ID NO: 20)

V82K 5′-CCCAGAAGACAAAAGAGGGAGCAGG-3′

(SEQ ID NO: 21)

A69K/V7OT/V71K/T72E 5′-GTGACAAATGTTGGAGGAAAAACAAAA
GAAGGTGTGACAGCAGTAGCC-3′

Construction of Vector for Production of Mutant α-Synuclein

Each pKF19k/mutant αsyn plasmid was digested with restriction enzymes NdeI and KpnI and ligated to the similarly-treated interin fusion expression vector pTYB1 to construct a vector for production of each mutant α-synuclein (hereinafter generally called as pTYB1/mutant αsyn). Escherichia coli DH5α-MCR was transformed with the vector in the same manner as for the wild type protein.

### Example 2

Preparation of Mutant Synuclein

Escherichia coli ER2566 having pTYB1/mutant α-syn was shake-cultured for one night at 37° C. in 450 ml of LB medium (final concentration of ampicillin: 100 μg/ml) in Sakaguchi's flask and inoculated into LB medium (7 L; containing 1 ml of Einol (antifoaming agent)) in a fermenter. Cultivation was started at 37° C. with aeration of 7 L/min. When OD₆₀₀ reached 0.5-0.8, IPTG was added at the final concentration of 0.3 mM to induce expression of α-synuclein fused to the intein-chitin binding domain. After starting the induction, temperature was lowered to 15° C. and incubation was continued for another 16 hours. The culture cells were collected by centrifugation (5,000 g, 4° C., 10 minutes) and the cells were washed twice with 0.85% NaCl.

Purification

After the incubation, the cells were collected, washed, and suspended in 20 mM Tris-HCl (pH 8.0), 1 mM EDTA and 50 mM NaCl. The cells were disrupted with a French press (110 MPa) and centrifuged (20,000×g, 4° C., 30 minutes). The supernatant from the centrifugation was applied to a chitin column (volume: about 10 ml) which was previously equilibrated with 20 mM Tris-HCl (pH 8.0), 1 mM EDTA and 500 mM NaCl. Non-adsorbed protein washed off with 20 mM Tris-HCl (pH 8.0), 1 mM EDTA and 0.1% Tween 20 in an amount of 10-fold of the column volume. Then 30 ml of 20 mM Tris-HCl (pH 7.4) was applied to the column to lower the salt concentration of the column. 30 ml of 20 mM Tris-HCl (pH 7.4) and 50 mM DTT was applied to the column and allowed to stand at 4° C. for 16 hours to allow for intein autolysis. Then 30 ml of 20 mM Tris-HCl (pH 7.4) was applied and the sample thus obtained was dialyzed against 20 mM Tris-HCl (pH 7.4) for three times. Finally, purity of the protein was confirmed by non-reduced SDS-PAGE.

### Example 3

Measurement of Changes in Structure of α-Synuclein by CD Spectrum

Purified α-synuclein was prepared in about 100 μg/ml and changes in the structure of α-synuclein upon temperature change were measured by CD spectrum. Temperature was changed from 3 to 90° C., and the spectrum was measured at 3° C., 15° C., 25° C., 40° C., 60° C. and 90° C. At each temperature point, spectra were measured for plural times to confirm that heat at that temperature was well transmitted to α-synuclein and the structure state reached constant. After that, the CD spectrum of the protein solution was determined. CD spectrum of the original buffer (20 mM Tris-HCl (pH 7.4), 50 mM NaCl) is subtracted from the CD spectrum of the protein solution, and the value was smoothed by means of a computer program. The ability for aggregation formation of the mutant α-synuclein was decreased as compared with the wild type α-synuclein.

### Example 4

Measurement of Changes in Structure by Fluorescence Probe

Purified α-synuclein was prepared in about 100 μg/ml and changes in the structure of α-synuclein upon temperature change were measured by fluorescent spectra. In the case of thioflavine T, 20 μM thioflavine T was added and measured at Ex 440 nm and Em 450-550 nm. In the case of 8-anilino-1-naphthalenesulfonic acid (ANS), 50 mM ANS was added and measured at Ex 380 nm and Em 400-600 nm. Temperature was changed from 3 to 90° C. and the spectrum was measured at 3° C., 15° C., 25° C., 40° C., 60° C. and 90° C. At each temperature point, spectra were measured for plural times to confirm that heat at that temperature was well transmitted to α-synuclein and the structure state reached constant. After that, the fluorescent spectrum of the protein solution was determined. The fluorescent spectrum of the original buffer (20 mM Tris-HCl (pH 7.4), 50 mM NaCl) was subtracted from the fluorescent spectrum of the protein solution, and the value was smoothed by means of a computer program. The ability for aggregation formation of the mutant α-synuclein was decreased as compared with the wild type α-synuclein.

### Example 5

Analysis of Aggregate Formation and Fibril Formation of the Mutant α-Synuclein

The purified wild type α-synuclein, the mutant α-synuclein constructed in the present invention and two types of mutant α-synucleins—Ala30Pro and Ala53Thr—found in patients suffering from familial Parkinson's disease were prepared in about 2 mg/ml and incubated at 37° C. Aliquot of 10 μl was collected in predetermined intervals. To the collected sample was added a buffer solution of 10 mM Tris-HCl, pH 7.4, containing a fluorescent dye thioflavine T (TfT) which specifically binds to a fibril structure in a final concentration of 25 μM to make a volume of 100 μl, and fluorescence spectrum was monitored immediately (Ex 440 nm; Em 450-550 nm). The fibril formation rate and the amount thereof were determined from the increase in the fluorescence intensity of TfT. This method has been commonly used for monitoring fibril formation caused by aggregation of a protein, such as amyloid.

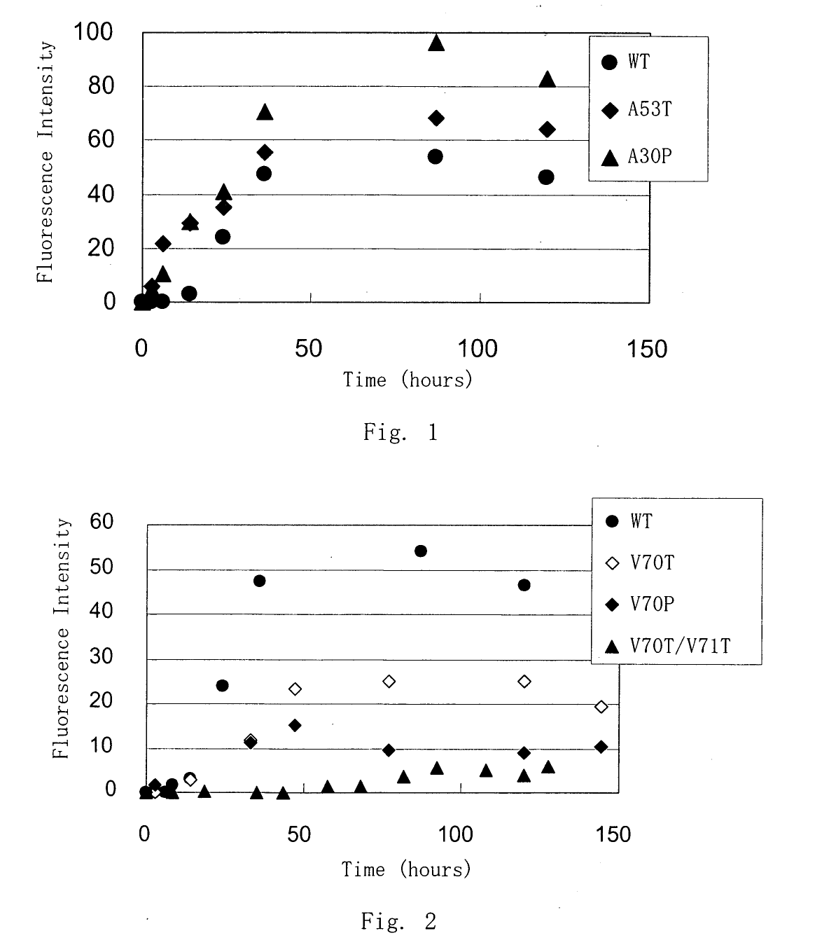

In the case of the two mutant α-synucleins—Ala30Pr and Ala53Thr—found in patients suffering from familial Parkinson's, the intensity of fluorescence significantly increased immediately after starting incubation of the sample, indicating that fibril was formed. In the case of wild type α-synuclein, the intensity of fluorescence increased after 12 hors. After that, fibril formation of those three types of α-synucleins proceeded and extensive fibril formation were observed as indicated by the fluorescence intensity of more than 50. The result is shown in FIG. 1. On the contrary, the mutant α-synucleins constructed by the present invention Val70Thr and Val70Pro and Val70Thr/Val71Thr containing two amino acid substitutions showed decreased ability of fibril formation. As shown in FIG. 2, the fibril formation rate of Val70Thr and Val70Pro mutant α-synucleins was not more than about 50% of that of the wild type. The final fibril formation amount of Val70Thr and Val70Pro was about 50% and about 20% of the wild type, respectively.

Further, mutant α-synucleins containing V74T, V77T or V82K substitution, four substitutions of A69K/V70T/V71K/T72E and five substitutions of A69K/V70T/V71K/T72E/V82K showed a fibril formation ability in nearly the same degree as that of Val71Thr. The fibril formation ability was about 50% of that of the wild type α-synuclein in terms of the formation rate and the final amount.

More surprisingly, little fibril formation ability was observed in Val70Thr/Val71Thr containing two substitutions. The fibril formation amount after 100 hours was not more than 10% of that of the wild type. As such, these mutant α-synucleins showed decreased fibril formation ability as compared with the wild type α-synuclein.

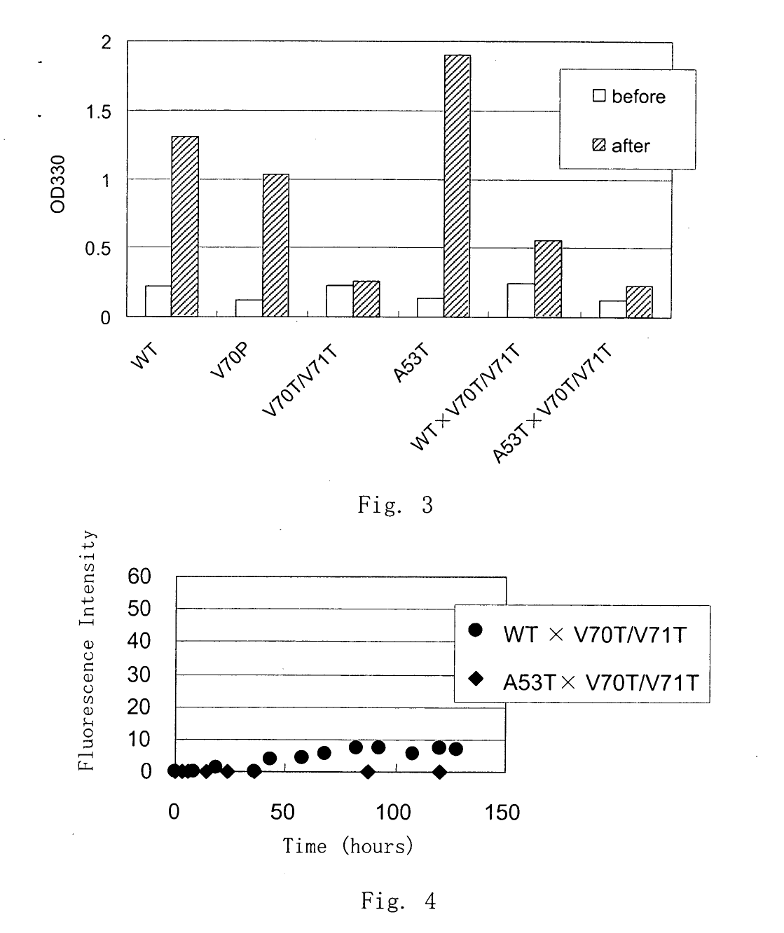

The total amount (sum of fibrillar and non-fibrillar components) of aggregate of α-synuclein formed in the solution of this experiment was evaluated by measuring the turbidity of the solution by way of scattering at 330 nm. The result is shown in FIG. 3. In the wild type, it was observed that aggregate was significantly formed as compared to those before incubation. It was further observed that more aggregates were formed than in the wild type in the mutant α-synuclein Ala53Thr found in patients suffering from familial Parkinson's disease. On the contrary, the ability of forming aggregate was decreased in the mutant α-synucleins constructed by the present invention Val70Pro and Val70Thr/Val71Thr containing two amino acid substitutions. The amount of the aggregate of Val70Pro mutant α-synuclein was not more than about 80% of that of the wild type. More surprisingly, the amount of aggregates of Val70Thr/Val71Thr containing two substitutions was about 15% of that of the wild type. As such, those mutant α-synucleins showed a decreased aggregation forming ability as compared with the wild type α-synuclein.

### Example 6

Inhibition of Aggregate Formation and Fibril Formation by Mutant α-Synuclein of Wild Type and of Mutant α-Synuclein Ala53Thr Found in Patients of Familial Parkinson's Disease

Purified Val70Thr/Val71Thr mutant α-synuclein (1 mg/ml) constructed in the present invention was mixed with the wild type α-synuclein or the mutant α-synuclein Ala53Thr found in patients suffering from familial Parkinson's disease. The mixture was adjusted to the total protein concentration of 2 mg/ml and incubated at 37° C. Aliquot of 10 μl was collected in predetermined intervals. To the collected sample was added a buffer solution of 10 mM Tris-HCl, pH 7.4, containing a fluorescent dye thioflavine T (TfT), which specifically binds to a fibril structure, in a final concentration of 25 μM to make a volume of 100 μl, and fluorescence spectrum was monitored immediately (Ex 440 nm; Em 450-550 nm). The fibril formation rate and the amount thereof were determined from the increase in the fluorescence intensity of TfT.

Even though the wild type α-synuclein or the mutant α-synuclein Ala53Thr found in patients suffering from familial Parkinson's disease was present, little fibril formation was observed as measured by TfT. The maximum fibril formation amount was reached after about 48 hours when the wild type was incubated alone, while in the mixture of the wild type α-synuclein and Val70Thr/Val71Thr, no fibril was formed at all at the same time as above. Even after incubation for 100 hours, the amount was not more than about 15% as compared with the case where the wild type was incubated alone (FIG. 4). Further, when the mutant α-synuclein Al53Thr found in patients suffering from familial Parkinson's disease was incubated alone, fibril was formed immediately after starting incubation and the maximum fibril formation amount was reached after 48 hours. On the contrary, in the mixture of the mutant α-synuclein Ala53Thr found in patients suffering from familial Parkinson's disease and Val70Thr/Val71Thr containing two substitutions which was constructed in the present invention, no fibril was formed at all even after incubation for 125 hours.

As observed above, the mutant α-synuclein constructed in the present invention was shown to inhibit the fibril formation of the wild type α-synuclein and of the mutant α-synuclein found in patients suffering from familial Parkinson's disease. Further, the total amount (sum of fibrillar and non-fibrillar components) of aggregate of α-synuclein formed in the solution in this experiment was evaluated by measuring the turbidity of the solution by way of scattering at 330 nm (FIG. 3). In the case the wild type or the mutant α-synuclein Ala53Thr which is found in patients suffering from familial Parkinson's disease was used alone, it was observed that a significant amount of aggregate was formed, while in the mixture of the wild type or Ala53Thr and Val70Thr/Val71Thr containing two substitutions which was constructed in the present invention, formation of aggregate was significantly decreased. Thus, it was shown that the mutant α-synuclein constructed in the present invention inhibited the ability of the wild type α-synuclein and of the mutant α-synuclein found in patients suffering from familial Parkinson's disease to form aggregate.

The above results suggested that the mutant α-synuclein constructed in the present invention is an effective therapeutic agent for treatment of various synucleopathy neurodegenerative diseases, such as Parkinson's disease, which are caused by fibril formation and aggregate formation of α-synuclein, and that α-synuclein is a key molecule for the development of novel therapeutic agents.

### Example 7

Inhibition of Fibril Formation of Wild Type α-Synuclein by Partial Structure Peptide

A synthetic α-synuclein partial structure peptide consisting of 10 amino acid residues of NH₂-Val-Gly-Gly-Ala-Thr-Thr-Thr-Gly-Val-Thr-COOH was dissolved at 0.2 mg/ml in a 2 mg/ml solution of purified wild type α-synuclein, and incubated at 37° C. Aliquot of 10 μl was collected in predetermined intervals. To the collected sample was added a buffer solution of 10 mM Tris-HCl, pH 7.4, containing a fluorescent dye thioflavine T (TfT), which specifically binds to a fibril structure, in a final concentration of 25 μM to make a volume of 100 μl, and fluorescence spectrum was monitored immediately (Ex 440 nm; Em 450-550 nm). The fibril formation rate and the amount thereof were determined from the increase in the fluorescence intensity of TfT.

The α-synuclein partial structure peptide was able to decrease the fibril formation ability of the wild type α-synuclein to the extent of about 20%, suggesting that the peptide has an anti-fibril formation ability.

### INDUSTRIAL APPLICABILITY

The mutant human α-synuclein of the present invention with decreased ability of forming aggregation is useful for investigation of pathology and treatment of Parkinson's disease and for research and development of gene therapy.

1. A mutant human α-synuclein having decreased aggregation forming ability.

2. A mutant human α-synuclein having the amino acid sequence comprising at least one of the following amino acid substitution in the amino acid sequence set forth in SEQ ID NO: 1: Gly68; Ala69; Val70; Val71; Thr72; Val74; Val77; and Val82.

3. A mutant human α-synuclein having the amino acid sequence which comprises at least one of the following amino acid substitutions in the amino acid sequence set forth in SEQ ID NO: 1:
substitution of Gly68 with threonine or valine;
substitution of Ala69 with threonine, valine or lysine
substitution of Val70 with threonine, proline or phenylalanine;
substitution of Val71 with threonine or lysine;
substitution of Thr72 with valine or glutamic acid;
substitution of Val74 with threonine;
substitution of Val77 with threonine; and
substitution of Val82 with lysine.

4. A mutant human α-synuclein comprising the amino acid substitutions Ala69Lys/Val70Thr/Val71Lys/Thr72Glu in the amino acid sequence set forth in SEQ ID NO: 1.

5. A mutant human α-synuclein comprising the amino acid substitutions Ala69Lys/Val70Thr/Val71Lys/Thr72Glu and Val82Lys in the amino acid sequence set forth in SEQ ID NO: 1.

6. A gene coding for the mutant human α-synuclein claimed in any one of claims 1 to 5.

7. A recombinant plasmid comprising the gene claimed in claim 6 introduced therein.

8. A transformant transformed with the recombinant plasmid claimed in claim 7.

9. A process for producing a mutant human α-synuclein comprising the steps of:
(a) introducing the gene claimed in claim 6 into a plasmid to prepare a recombinant plasmid;
(b) transforming a host with the recombinant plasmid of (a) to prepare a transformant; and
(c) culturing the transformant of (b) to produce the mutant human α-synuclein.

10. A composition for inhibiting aggregation of the wild type human α-synuclein, Ala53Thr mutant human α-synuclein or Ala50Pro mutant human α-synuclein, comprising the mutant human α-synuclein claimed in any one of claims 1 to 5.

11. A method for inhibiting aggregation of the wild type human α-synuclein, Ala53Thr mutant human α-synuclein or Ala50Pro mutant human α-synuclein in a cell, tissue or organism, comprising contacting the cell, tissue or organism with the mutant human α-synuclein claimed in any one of claims 1 to 5.

12. A peptide having a sequence of 10 or more contiguous amino acid residues in the following amino acid sequence:
Gln-Val-Thr-Asn-Val-Gly-Gly-Ala-Thr-Thr-Thr-Gly-Val-Thr-Ala-Val-Ala-Gln (SEQ ID NO: 22).

13. A peptide having the following amino acid sequence:
Val-Gly-Gly-Ala-Thr-Thr-Thr-Gly-Val-Thr (SEQ ID NO: 23).

14. A composition for inhibiting aggregation of the wild type human α-synuclein, Ala53Thr mutant human α-synuclein or Ala50Pro mutant human α-synuclein, comprising the peptide claimed in claim 12 or 13.

15. A method for inhibiting aggregation of the wild type human α-synuclein, Ala53Thr mutant human α-synuclein or Ala50Pro mutant human α-synuclein in a cell, tissue or organism, comprising contacting the cell, tissue or organism with the peptide claimed in claim 12 or 13.


SEQUENCE LISTING (23 sequences)

SEQ ID NO: 1 - protein, 140 aa, Homo sapiens
MDVFMKGLSKAKEGVVAAAEKTKQGVAEAAGKTKEGVLYVGSKTKEGVVHGVATVAEKTKEQVTNVGGAVVTGVTAVAQKTVEGAGSIAAATGFVKKDQLGKNEEGAPQEGILEDMPVDPDNEAYEMPSEEGYQDYEPEA

SEQ ID NO: 2 - DNA, 423 bp, Homo sapiens
atggatgtattcatgaaaggactttcaaaggccaaggagggagttgtggctgctgctgagaaaaccaaacagggtgtggcagaagcagcaggaaagacaaaagagggtgttctctatgtaggctccaaaaccaaggagggagtggtgcatggtgtggcaacagtggctgagaagaccaaagagcaagtgacaaatgttggaggagcagtggtgacgggtgtgacagcagtagcccagaagacagtggagggagcagggagcattgcagcagccactggctttgtcaaaaaggaccagttgggcaagaatgaagaaggagccccacaggaaggaattctggaagatatgcctgtggatcctgacaatgaggcttatgaaatgccttctgaggaagggtatcaagactacgaacctgaagcctaa

SEQ ID NO: 3 - DNA, 37 bp, Artificial Sequence
  Description of Artificial Sequence Synthetic PCR primer
cgccatatggatgtattcatgaaaggactttcaaagg

SEQ ID NO: 4 - DNA, 42 bp, Artificial Sequence
  Description of Artificial Sequence Synthetic PCR primer
ggtacccttggcaaagcaggcttcaggttcgtagtcttgata

SEQ ID NO: 5 - DNA, 36 bp, Artificial Sequence
  Description of Artificial Sequence Synthetic PCR primer
ccatggatgtattcatgaaaggactttcaaaggcca

SEQ ID NO: 6 - DNA, 37 bp, Artificial Sequence
  Description of Artificial Sequence Synthetic PCR primer
cctgcagtatttcttaggcttcaggttcgtagtcttg

SEQ ID NO: 7 - DNA, 36 bp, Artificial Sequence
  Description of Artificial Sequence Synthetic PCR primer
ccaagcttcatatggatgtattcatgaaaggacttt

SEQ ID NO: 8 - DNA, 42 bp, Artificial Sequence; identical to SEQ ID NO: 4
  Description of Artificial Sequence Synthetic PCR primer

SEQ ID NO: 9 - DNA, 25 bp, Artificial Sequence
  Description of Artificial Sequence Synthetic PCR primer
caaatgttggaacagcagtggtgac

SEQ ID NO: 10 - DNA, 25 bp, Artificial Sequence
  Description of Artificial Sequence Synthetic PCR primer
caaatgttggagtggcagtggtgac

SEQ ID NO: 11 - DNA, 23 bp, Artificial Sequence
  Description of Artificial Sequence Synthetic PCR primer
gttggaggaacagtggtgacggg

SEQ ID NO: 12 - DNA, 23 bp, Artificial Sequence
  Description of Artificial Sequence Synthetic PCR primer
gttggaggagtggtggtgacggg

SEQ ID NO: 13 - DNA, 22 bp, Artificial Sequence
  Description of Artificial Sequence Synthetic PCR primer
ggaggagcaacagtgacgggtg

SEQ ID NO: 14 - DNA, 22 bp, Artificial Sequence
  Description of Artificial Sequence Synthetic PCR primer
ggaggagcacctgtgacgggtg

SEQ ID NO: 15 - DNA, 22 bp, Artificial Sequence
  Description of Artificial Sequence Synthetic PCR primer
ggaggagcatttgtgacgggtg

SEQ ID NO: 16 - DNA, 39 bp, Artificial Sequence
  Description of Artificial Sequence Synthetic PCR primer
caaatgttggaggagcaacaacaacgggtgtgacagcag

SEQ ID NO: 17 - DNA, 24 bp, Artificial Sequence
  Description of Artificial Sequence Synthetic PCR primer
gagcagtggtggtgggtgtgacag

SEQ ID NO: 18 - DNA, 23 bp, Artificial Sequence
  Description of Artificial Sequence Synthetic PCR primer
ggtgacgggtacaacagcagtag

SEQ ID NO: 19 - DNA, 25 bp, Artificial Sequence
  Description of Artificial Sequence Synthetic PCR primer
gtgtgacagcaaccgcccagaagac

SEQ ID NO: 20 - DNA, 25 bp, Artificial Sequence
  Description of Artificial Sequence Synthetic PCR primer
cccagaagacaaaagagggagcagg

SEQ ID NO: 21 - DNA, 48 bp, Artificial Sequence
  Description of Artificial Sequence Synthetic PCR primer
gtgacaaatgttggaggaaaaacaaaagaaggtgtgacagcagtagcc

SEQ ID NO: 22 - protein, 18 aa, Artificial Sequence
  Description of Artificial Sequence Synthet ic peptide
QVTNVGGATTTGVTAVAQ

SEQ ID NO: 23 - protein, 10 aa, Artificial Sequence
  Description of Artificial Sequence Synthetic peptide
VGGATTTGVT